(19) United States
(12) Patent Application Publication
(10) Pub. No.: US 2010/0152830 A1
(43) Pub. Date: Jun. 17, 2010

(54) Single Wire Stent Delivery System

(75) Inventors: Jan Weber, Maastricht (NL); James Anderson, Fridley, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: BOSTON SCIENTIFIC SCIMED, INC., Maple Grove, MN (US)

(21) Appl. No.: 12/615,704

(22) Filed: Nov. 10, 2009

Related U.S. Application Data
(60) Provisional application No. 61/122,969, filed on Dec. 16, 2008.

(51) Int. Cl. A61F 2/84

(52) U.S. Cl. 623/1.11

(57) ABSTRACT

A stent delivery system for deployment at a bifurcated vessel includes a catheter and a balloon. The length of the balloon is defined by a proximal balloon region and a distal balloon region. The proximal balloon region is disposed about a first longitudinal axis and the distal balloon region is disposed about a second longitudinal axis. The balloon has an unexpanded state and an expanded state, and a bend along its length. The proximal balloon region intersects the distal balloon region at the bend. In both the unexpanded state and the expanded state, at least one of the first longitudinal axis and the second longitudinal axis define an oblique angle relative to the main vessel longitudinal axis.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates to implantable medical devices, such as stents and similar endoprostheses, and particularly to those configured for use at a vessel bifurcation. Some embodiments of the invention are directed to delivery systems, such as catheter systems of all types, which are utilized in the delivery of such devices.

2. Description of the Related Art

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system. They may be self-expanding, expanded by an internal radial force, such as when mounted on a balloon, or a combination of self-expanding and balloon expandable (hybrid expandable).

Within the vasculature it is not uncommon for stenoses to form at a vessel bifurcation. A bifurcation is an area of the vasculature or other portion of the body where a first (or parent) vessel is bifurcated into two or more tubular component vessels. Where a stenotic lesion or lesions form at such a bifurcation, the lesion(s) can affect only one of the vessels (i.e., either of the tubular component vessels or the parent vessel) two of the vessels, or all three vessels.

Many of the bifurcated stents that have been disclosed include a primary branch and at least one secondary (side) branch which is positioned adjacent to and/or partially within the primary branch. Often such systems employ multiple guide wires in order to properly advance the catheter to the vessel bifurcation and properly align the secondary branch with the ostium of the side branch vessel.

Given that most vessel bifurcations are asymmetric, proper positioning and alignment of the stent within the vessel bifurcations is a necessity. Procedural time however, is a critical component in the long term successful outcome of the deployment procedure, and systems that require multiple guide wires (each of which needing careful and precise positioning) unavoidably increase the time the procedure takes. As such a need to simplify and reduce the procedure time exists. The present invention addresses this need by providing an elegant and robust single wire approach to procedures involving the deployment of stent(s) at vessel bifurcations.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

In at least one embodiment, the invention is directed to a stent delivery system for deployment at a bifurcated vessel. The bifurcated vessel comprises a main vessel, a first branch vessel, and a second branch vessel. The main vessel defines a main vessel longitudinal axis. The delivery system comprises a catheter having a catheter shaft, and a balloon. The balloon is engaged to and disposed about a distal portion of the catheter shaft. The length of the balloon is defined by a proximal balloon region and a distal balloon region. The proximal balloon region is disposed about a first longitudinal axis and the distal balloon region is disposed about a second longitudinal axis. The balloon has an unexpanded state and an expanded state, and the balloon has a bend along its length. The proximal balloon region intersects the distal balloon region at the bend. In both the unexpanded state and the expanded state, one or both of the first longitudinal axis and the second longitudinal axis define an oblique angle relative to the main vessel longitudinal axis.

In some embodiments, the present invention further comprises a stent. The stent has a proximal end and a distal end, the stent being disposed about the balloon. The stent has a generally tubular shape and is comprised of a plurality of generally tubular bands. The stent has a first stent region and a second stent region. The stent further has a deployed state and an undeployed state, and in both the deployed state and the undeployed state the first stent region is disposed about the first longitudinal axis and the second stent region being is about the second longitudinal axis. The second branch vessel has an ostium. The first stent region and the second stent region form an elbow, the elbow being positioned between the proximal end of the stent and the distal end of the stent. In the deployed state, the proximal end of the stent is positioned substantially within the main vessel, the distal end of the stent is positioned substantially within the first branch vessel, and the elbow is positioned adjacent the ostium of the second branch vessel.

In at least one embodiment, the stent further comprises deployable elements engaged to a perimeter. In the deployed state the first stent region defines a first lumen, the second stent region defines a second lumen, and the deployable elements define a third lumen. The third lumen has a third longitudinal axis, the third longitudinal axis being different from the first longitudinal axis and the second longitudinal axis.

These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a perspective view of a stent delivery system, in accordance with at least one embodiment of the present invention.

FIG. 2 is the stent delivery system of FIG. 1, shown in an inflated state, in accordance with at least one embodiment of the present invention.

FIG. 3 is a cross-sectional perspective view of the stent delivery system of FIG. 1, shown in a bifurcated vessel, in accordance with at least one embodiment of the present invention.

FIG. 4 is a cross-sectional perspective view of the stent of FIG. 3, shown deployed at the bifurcated vessel, in accordance with at least one embodiment of the present invention.

FIG. 5 is a perspective view of the stent delivery system of FIG. 2, shown with its petals deployed, in accordance with at least one embodiment of the present invention.

FIGS. 6a-6c are cross-sectional perspective views of the stent delivery system of FIG. 1 depicting deployment of its petals at the bifurcated vessel, in accordance with at least one embodiment of the present invention.

FIG. 7a is a cross-sectional perspective view of a stent delivery system, wherein the elbow is placed closer to the distal end of the stent then the proximal end, in accordance with at least one embodiment of the present invention.

FIG. 7b is a cross-sectional perspective view of a stent delivery system, wherein the elbow is placed closer to the proximal end of the stent then the distal end, in accordance with at least one embodiment of the present invention.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

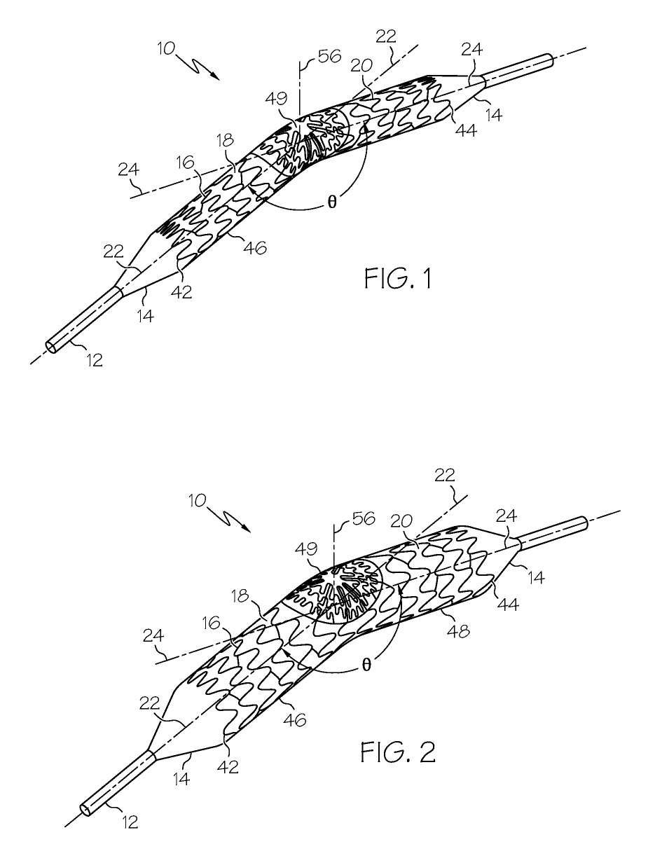

Referring now to FIG. 1, a stent delivery system 10 is shown, in accordance with at least one embodiment of the present invention. FIG. 1 depicts the delivery system 10 in an uninflated state with catheter 12, balloon 14, and stent 16. As seen in FIG. 1, the inflatable balloon is disposed about the catheter, and the balloon has two distinct regions: a first balloon region 18 and a second balloon region 20. The first balloon region 18 is disposed about a first longitudinal axis 22 and the second balloon region 20 is disposed about a second longitudinal axis 24. The first longitudinal axis is different from the second longitudinal axis, and as illustrated in FIGS. 1 and 2, the first longitudinal axis and the second longitudinal axis are non-parallel. Furthermore, the first longitudinal axis 22 and the second longitudinal axis 24 form an oblique angle θ with one another. The term “oblique” as used herein is defined as “an angle that is neither zero nor 180 degrees, and which may be a 90 degree angle.”

FIG. 2 depicts the stent delivery system 10 of FIG. 1 in an inflated state. In the inventive stent delivery system 10, the balloon 14 maintains an oblique angle θ between the first balloon region 18 and the second balloon region 20, despite the fact that the balloon has been fully inflated.

Balloons molded in a straight geometry often straighten out at high pressures. In the embodiment depicted in FIGS. 1 and 2 however, the catheter 10 is provided with a balloon 14 that has an imposed or manufactured bend that is maintained even under pressure. In some embodiments, the system 10 utilizes a heated crimper to force a bent shape into the balloon during balloon manufacture. This process, or a process of manufacturing a balloon using a curved balloon mold, allows the balloon to retain a bend in its shape in both the uninflated and inflated states.

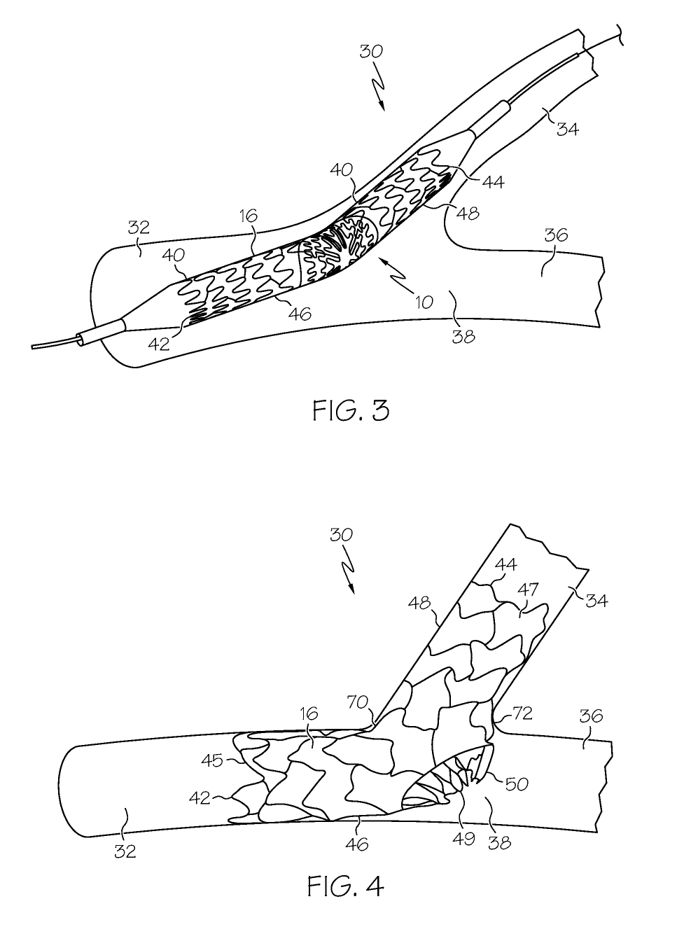

It has been found that by including an angle in the balloon, stent delivery is simplified. The rotational alignment of the stent delivery system is improved by rotating an angled balloon, with a shape as in FIGS. 1 and 2, into a side branch. As the catheter is advanced through a main vessel, the proximal region 20 of the balloon will tend to rotate relative to the axis 22 of the first region. Such rotation will naturally tend to encourage the second (distal) region 20 to enter into a side branch vessel 34 of a bifurcation 30 of the main vessel 32 (see FIG. 3). Such tendency will also act to position the bend of the balloon (“the bend” being a portion of the balloon wherein the first region and second region intersect) across the ostium of the adjacent branch vessel of the bifurcation while the first region 18 of the balloon remains positioned in the main vessel.

In at least one embodiment, the balloon maintains an angle even when a pressure of 18 atm is exerted. There is a significant angle in both inflated and uninflated states. Bended crimpers, such as those described in U.S. Pat. No. 7,207,204, the entire contents of which is expressly incorporated herein by reference, can be used to produce an angled balloon.

Referring now to FIG. 3, a bifurcated vessel 30 is shown having main branch vessel 32, first branch vessel 34, and second branch vessel 36 having an ostium, or opening, 38. The stent delivery system 10 of FIG. 1 has been delivered to the bifurcation site. As seen in FIG. 3, the stent 16 is made up of generally tubular bands 40 and has a generally tubular shape with a proximal end 42 and a distal end 44. Referring again to FIGS. 1 and 2, the stent 16 has a first stent region 46 disposed about the first longitudinal axis 22 and a second stent region 48 disposed about the second longitudinal axis 24.

As best seen in FIGS. 1 and 2, an elbow 49 is formed at the region in which the first stent region 46 joins the second stent region 48. The elbow 49 is positioned between the proximal and distal ends (42, 44) of the stent. In some embodiments, for example when the elbow is positioned substantially equidistant between the proximal and distal ends, the angle may be about 10 degrees to about 30 degrees. In at least one embodiment, for example when the elbow is closer to one of the ends, the angle may be an angle up to about 45 degrees.

Referring now to FIG. 4, the stent 16 is shown in a deployed state. A first lumen 45 is defined by the first stent region 46, and a second lumen 47 is defined by the second stent region 48. In the deployed state, the proximal end 42 of the stent is positioned substantially within the main branch 32, the distal end 44 of the stent is positioned substantially within the first branch vessel 34, and the elbow 49 is positioned adjacent to, or within, the ostium 38 of the second branch vessel 36. It should be further noted that in some embodiments, such as shown in FIG. 4, the first stent region 46 is positioned substantially within the main branch 32, the second stent region 48 is positioned substantially within the first branch vessel 34, and the elbow 49 is positioned adjacent to, or within, the ostium 38 of the second branch vessel 36.

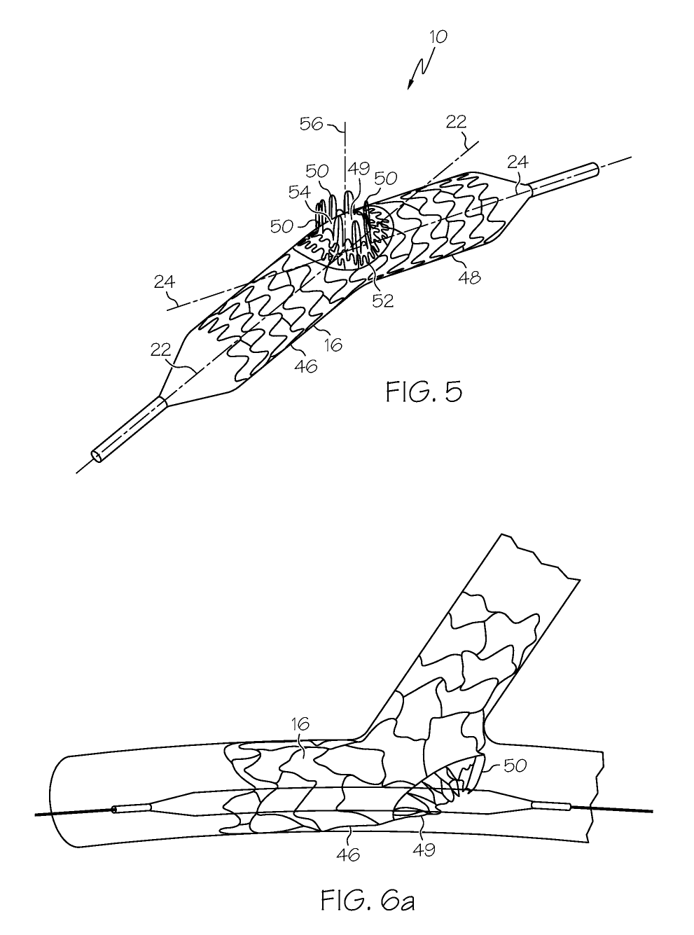

Referring now to FIG. 5, some embodiments of the stent delivery system 10 include deployable elements 50, or petals, adjacent to or surrounding the elbow 49. In some embodiments, the petals are engaged to a perimeter 52 that encircles the elbow 49, as seen in FIG. 5. After the stent 16 is deployed at the bifurcation site, the petals are deployed outwardly. Examples of bifurcated stents with petals can be found in U.S. Application Publication No. 2007/0225796, the entire contents of which being expressly incorporated herein by reference.

Still referring to FIG. 5, when petals 50 are deployed the petals 50 define a third lumen 54 having a third longitudinal axis 56. The third longitudinal axis 56 is different from and non-parallel to both the first longitudinal axis 22 and the second longitudinal axis 24.

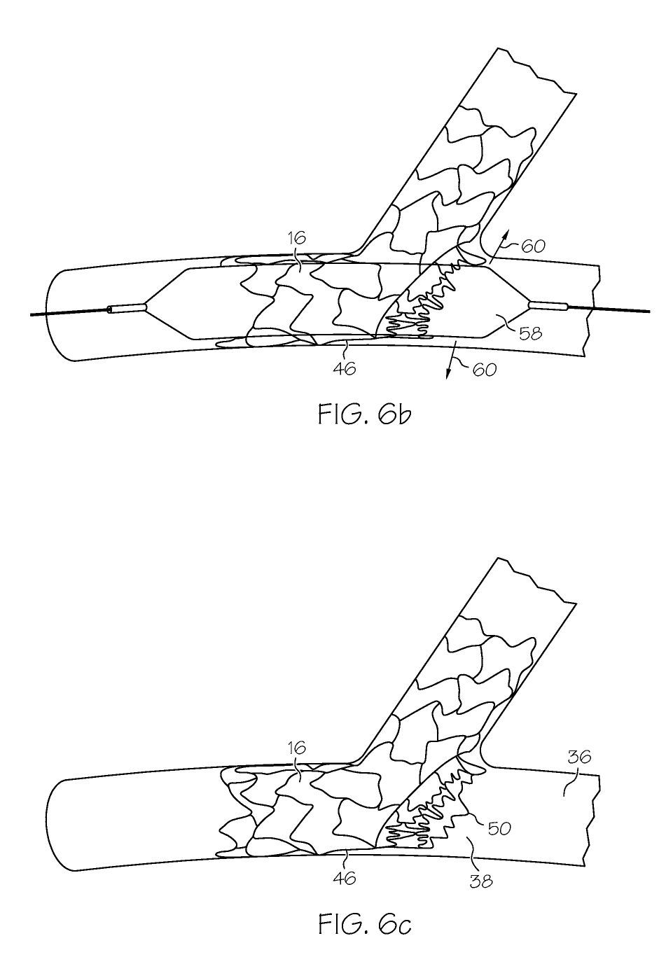

In at least one embodiment, the petals are designed to deploy and expand outwardly through the use of a secondary balloon, as seen in FIGS. 6a-6c. FIG. 6a shows an uninflated secondary balloon 58 inserted through the first stent region 46 and the elbow 49 of the stent 16. The balloon 58 is inflated such that radially outward forces 60 push the petals outwardly, as in FIG. 6b. FIG. 6c illustrates the stent 16 of FIG. 4 with the petals 50 fully deployed adjacent the ostium 38 of the secondary vessel 36.

In some embodiments, the petals are designed to be self-expandable so as to deploy and expand outwardly without the need for a secondary balloon. In some embodiments the petals 50 deploy into the lumen of the secondary vessel 36 during initial balloon deployment of the first stent region 46 and second stent region 48.

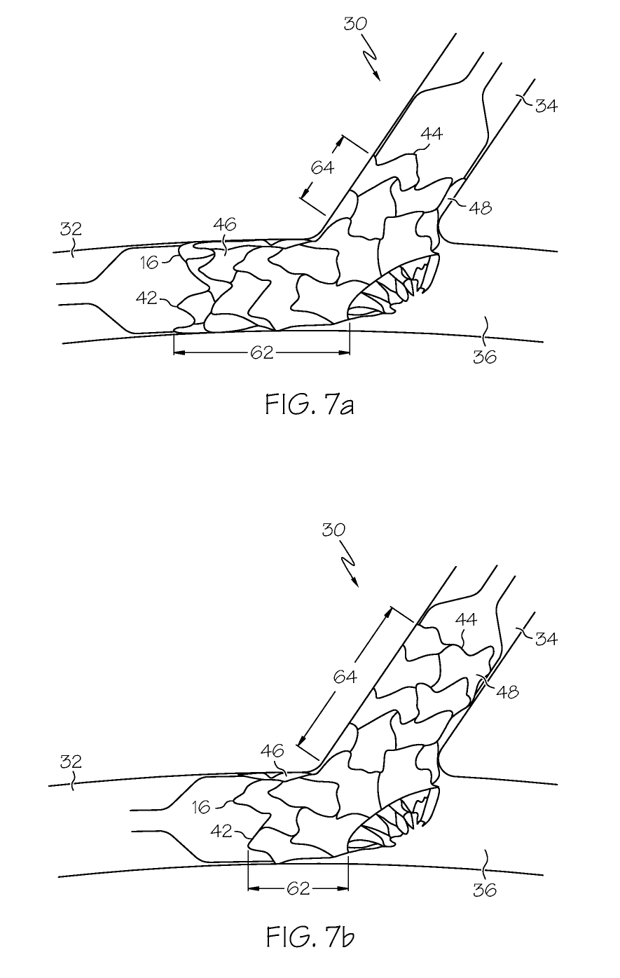

Referring now to FIG. 7a, a stent 16 is depicted where the elbow is positioned closer to the distal end of the stent than the proximal end of the stent. That is, the first stent region 46 has a first length 62 and the second stent region 48 has a second length 64, the first length being greater than the second length. Similarly, in FIG. 7b, the elbow is positioned closer to the proximal end of the stent than the distal end of the stent. That is, the first stent region 46 has a first length 62 and the second stent region 48 has a second length 64, the first length being less than the second length.

In at least one embodiment of the present invention, the stent includes reinforced areas. For example, in FIG. 4, the areas of the stent 16 indicated at 70 and 72 may include reinforcement material, such as reinforcement longitudinal strips. The reinforcement material placed at area 70 may extend towards the proximal and distal ends (42, 44) of the stent as much as necessary in order to properly reinforce the stent 16. Similarly, the reinforcement material placed at area 72 may extend between the petals 50 and the distal end 44 as much as necessary in order to properly reinforce the stent 16.

In some embodiments of the present invention, it may be desirable to coat the balloon with a therapeutic agent(s). In at least one embodiment, some portions of the balloon may include extra drug coating. For example, an elbow is also formed at the region in which the first balloon region joins the second balloon region. The elbow has an outer portion and an inner portion. In some embodiments, the inner portion of the elbow includes extra therapeutic agent, while the outer portion of the elbow is not coated, or only minimally coated, with a therapeutic agent.

In some embodiments the at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. In some embodiments, the therapeutic agent comprises an immunosuppressant such as Sirolimus (rapamycin). Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

In some embodiments the stent, the delivery system, or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

What is claimed is:

1. A stent delivery system for deployment at a bifurcated vessel, the bifurcated vessel comprising a main vessel, a first branch vessel, and a second branch vessel, the main vessel defining a main vessel longitudinal axis, the delivery system comprising:
a catheter, the catheter having a catheter shaft; and
a balloon, the balloon engaged to and disposed about a distal portion of the catheter shaft, the balloon having a length, the length of the balloon defined by a proximal balloon region and a distal balloon region, the proximal balloon region disposed about a first longitudinal axis and the distal balloon region disposed about a second longitudinal axis,
the balloon having an unexpanded state and an expanded state, the balloon having a bend along the length, wherein the proximal balloon region intersects the distal balloon region at the bend,
in both the unexpanded state and the expanded state at least one of the first longitudinal axis and the second longitudinal axis defining an oblique angle relative to the main vessel longitudinal axis.

2. The stent delivery system of claim 1, further comprising a stent, wherein the stent has a proximal stent region and a distal stent region, the stent further having a deployed state and an undeployed state, in both the deployed state and the undeployed state the proximal stent region being disposed about the first longitudinal axis and the distal stent region being disposed about the second longitudinal axis.

3. The stent delivery system of claim 2, wherein the second branch vessel defines an ostium, and
wherein the proximal stent region and the distal stent region form an elbow, the elbow being positioned between a proximal end of the stent and a distal end of the stent, and
wherein in an initially deployed state the proximal end of the stent is positioned substantially within the main vessel, the distal end of the stent is positioned substantially within the first branch vessel, and the elbow is positioned adjacent the ostium of the second branch vessel.

4. The stent delivery system of claim 3 wherein in a fully deployed state the elbow defines a side branch region that extends into the second branch vessel, the side branch region being disposed about a side branch longitudinal axis, the side branch longitudinal axis forming an angle alpha with the first longitudinal axis, the side branch longitudinal axis forming an angle beta with the second longitudinal axis.

5. The stent delivery system of claim 4, wherein the angle alpha is an angle of about 10 degrees to about 30 degrees.

6. The stent delivery system of claim 4, wherein the angle beta is an angle up to about 45 degrees.

7. The stent delivery system of claim 4, the stent further comprising a perimeter element, the perimeter element encircling the elbow.

8. The stent delivery system of claim 7, further comprising deployable elements, the deployable elements being engaged to the perimeter, the deployable elements at least partially defining the side branch region,
wherein in the deployed state the first stent region defines a first lumen and the second stent region defines a second lumen, and
wherein in the fully deployed state the deployable elements define a third lumen, the third lumen having a third longitudinal axis, the third longitudinal axis being different from the first longitudinal axis and the second longitudinal axis.

9. The stent delivery system of claim 8, wherein the deployable elements are self-expandable.

10. The stent delivery system of claim 8, wherein the deployable elements are balloon expandable.

11. The stent delivery system of claim 8, wherein the first stent region has a first length and the second stent region has a second length, wherein the elbow is positioned closer to the distal end of the stent than the proximal end of the stent.

12. The stent delivery system of claim 8, wherein the first stent region has a first length and the second stent region has a second length, wherein the elbow is positioned closer to the proximal end of the stent than the distal end of the stent.

13. A stent delivery system for deployment at a bifurcated vessel, the bifurcated vessel comprising a main vessel, a first branch vessel, and a second branch vessel, the main vessel defining a main vessel longitudinal axis, the delivery system comprising:
a catheter, the catheter having a catheter shaft; and
a balloon, the balloon engaged to and disposed about a distal portion of the catheter shaft, the balloon having a length, the length of the balloon defined by a proximal balloon region and a distal balloon region, the proximal balloon region disposed about a first longitudinal axis and the distal balloon region disposed about a second longitudinal axis,
the balloon having an unexpanded state and an expanded state, the balloon having a bend along the length, wherein the proximal balloon region intersects the distal balloon region at the bend,
in both the unexpanded state and the expanded state at least one of the first longitudinal axis and the second longitudinal axis defining an oblique angle relative to the main vessel longitudinal axis; and
a stent, the stent having a proximal end and a distal end, the stent being disposed about the balloon, the stent having a generally tubular shape, the stent being comprised of a plurality of generally tubular bands, the stent having a first stent region and a second stent region, the stent further having a deployed state and an undeployed state, in both the deployed state and the undeployed state the first stent region being disposed about the first longitudinal axis and the second stent region being disposed about the second longitudinal axis,
wherein the second branch vessel has an ostium, and
wherein the first stent region and the second stent region form an elbow, the elbow being positioned between the proximal end of the stent and the distal end of the stent, and
wherein in the deployed state the proximal end of the stent is positioned substantially within the main vessel, the distal end of the stent is positioned substantially within the first branch vessel, and the elbow is positioned adjacent the ostium of the second branch vessel, and
wherein the stent further comprises deployable elements, the deployable elements being engaged to a perimeter, and
wherein in the deployed state the first stent region defines a first lumen, the second stent region defines a second lumen, and the deployable elements define a third lumen, the third lumen having a third longitudinal axis, the third longitudinal axis being different from the first longitudinal axis and the second longitudinal axis.

14. The stent delivery system of claim 13, wherein the perimeter element encircles the elbow.

15. The stent delivery system of claim 13, wherein the deployable elements are self-expandable.

16. The stent delivery system of claim 13, wherein the deployable elements are balloon expandable.
